(19) United States
(12) Patent Application Publication
(10) Pub. No.: US 2017/0055946 A1
(43) Pub. Date: Mar. 2, 2017

(54) ULTRASOUND PROBE AND ULTRASOUND DIAGNOSIS APPARATUS

(71) Applicant: KONICA MINOLTA, INC., Tokyo (JP)

(72) Inventor: Kiyoshi FUJII, Yokohama-shi (JP)

(73) Assignee: KONICA MINOLTA, INC., Tokyo (JP)

(21) Appl. No.: 15/242,451

(22) Filed: Aug. 19, 2016

(30) Foreign Application Priority Data
Sep. 2, 2015 (JP) 2015-172987

(51) Int. Cl. A61B 8/00; B29C 65/70; B29C 65/50; C25D 7/00; C23C 14/34; C23C 14/24; C23C 18/31; A61B 8/12; C23C 14/14

(52) U.S. Cl. CPC A61B 8/4461; A61B 8/12; A61B 8/4494; A61B 8/4281; B29C 65/70; B29C 65/505; C23C 14/14; C23C 14/34; C23C 14/24; C23C 18/31; C25D 7/00; B29K 2063/00

(57) ABSTRACT

An ultrasound probe includes: a drive motor of an outer rotor type; an ultrasound element configured to transmit ultrasound waves, and receive ultrasound waves; and a rotary transformer configured to transfer a signal of the ultrasound element in a non-contact manner, the rotary transformer being disposed at an end of the drive motor, wherein the drive motor includes: a stator including a stator core and a motor coil; and a rotor including a rotor core and a motor magnet, the ultrasound element is attached to the rotor, the rotary transformer includes a secondary transformer disposed on the rotor, and a primary transformer disposed on the opposite side from the secondary transformer, the prmary transformer and the secondary transformer each include a transformer core, and a transformer coil disposed on the transformer core, and at least one of the primary transformer and the secondary transformer includes a coil holding portion.

The entire disclosure of Japanese Patent Application No. 2015-172987 filed on Sep. 2, 2015 including description, claims, drawings, and abstract are incorporated herein by reference in its entirety.

### BACKGROUND OF THE INVENTION

Field of the Invention

The present invention relates to an ultrasound probe of a mechanical sector scanning type, and an ultrasound diagnosis apparatus.

Description of the Related Art

One of the known medial apparatuses is an ultrasound diagnosis apparatus that visualizes and examines tissues and organs in a living organism with ultrasound waves. Such an ultrasound diagnosis apparatus includes an ultrasound probe and a main diagnosis device. The ultrasound probe transmits ultrasound waves toward an object, receives reflected ultrasound waves, converts the reflected ultrasound waves into electrical signals (hereinafter referred to as “ultrasound signals”), and transmits the ultrasound signals to the main diagnosis device. The main diagnosis device supplies a drive signal to the ultrasound probe, and generates and displays a diagnostic image (a tomographic image) in accordance with the received ultrasound signals.

Examples of scanning methods for obtaining diagnostic images include a mechanical sector scanning method by which scanning is performed while an ultrasound element (an ultrasound transducer) in an ultrasound probe is mechanically rotated, for example. The ultrasound probe of the mechanical sector scanning type includes a drive motor for rotating an ultrasound element. The ultrasound element is attached to the rotor of the drive motor of an outer rotor type, for example, and rotates with the rotor.

The ultrasound probe also includes a signal transfer path for transferring ultrasound signals from the ultrasound element to the main diagnosis device. A rotary transformer is normally used as part of the signal transfer path (see JP 2002-301081 A and JP 2002-345822 A, for example). As a rotary transformer is used, signal transfer can be performed in a non-contact manner, and thus, rotation of the rotor can be prevented from being hindered by a signal wire.

An ultrasound diagnosis apparatus is expected to generate high-definition diagnostic images with less noise. In view of this, various noise prevention measures, such as a shield, have been taken. When the inventors experimentally produced an ultrasound probe of the mechanical sector scanning type, however, it became clear that the conventional noise prevention measures were not sufficient, and there was room for improvement.

In view of the above, the inventors paid attention to the rotary transformer forming part of the signal transfer path in an ultrasound probe, and made intensive studies to reduce noise. As a result, the inventors discovered that noise appeared, because the transformer coil forming the rotary transformer was affected by flux leakage from the motor magnet and vibrated at a time of transmission/reception of ultrasound waves. The present invention has been completed on the basis of these findings.

### SUMMARY OF THE INVENTION

An object of the present invention is to provide an ultrasound probe and an ultrasound diagnosis apparatus that can reduce noise caused by electromagnetic force generated by the interaction between flux leakage from a motor magnet and the current flowing in a transformer coil, and obtain high-definition diagnostic images.

To achieve the abovementioned object, according to an aspect, an ultrasound probe reflecting one aspect of the present invention comprises:

a drive motor of an outer rotor type;

an ultrasound element configured to transmit ultrasound waves toward an object, and receive ultrasound waves reflected by the object; and

a rotary transformer configured to transfer a signal of the ultrasound element in a non-contact manner, the rotary transformer being disposed at an end of the drive motor in a direction of rotational axis,

wherein

the drive motor includes: a stator including a stator core and a motor coil; and a rotor including a rotor core and a motor magnet,

the ultrasound element is attached to the rotor,

the rotary transformer includes a secondary transformer disposed on the rotor, and a primary transformer disposed on the opposite side from the secondary transformer,

the primary transformer and the secondary transformer each include a transformer core, and a transformer coil disposed on the transformer core, and

at least one of the primary transformer and the secondary transformer includes a coil holding portion configured to maintain a winding state of the transformer coil.

To achieve the abovementioned object, according to an aspect, an ultrasound diagnosis apparatus reflecting one aspect of the present invention comprises:

the ultrasound probe described above; and

a main diagnosis device configured to supply a drive signal to the ultrasound probe, and generate a diagnostic image in accordance with an ultrasound signal from the ultrasound probe, the main diagnosis device being connected to the ultrasound probe.

### BRIEF DESCRIPTION OF THE DRAWINGS

The above and other objects, advantages and features of the present invention will become more fully understood from the detailed description given hereinbelow and the appended drawings which are given by way of illustration only, and thus are not intended as a definition of the limits of the present invention, and wherein:

FIG. 1 is an external view of an ultrasound diagnosis apparatus according to an embodiment of the present invention;

FIG. 2 is a diagram schematically showing the structure of the tip portion of an ultrasound probe;

FIG. 3 is a cross-sectional view of the inner structure of the tip portion of the ultrasound probe;

FIG. 4 is a cross-sectional view of the inner structure of the tip portion of the ultrasound probe;

FIG. 5 is a diagram showing a piezoelectric plate and a transformer col in a connected state;

FIG. 6 is a plan view of a primary transformer;

FIG. 7 is a perspective view of a transformer core; and

FIG. 8 is a diagram showing another example of rotary transformers.

### DESCRIPTION OF THE PREFERRED EMBODIMENTS

In a conventional rotary transformer, the transformer coil is not firmly secured to the transformer core. For example, where a groove is formed in the transformer core, and the transformer coil is disposed in the groove and is partially bonded to the transformer core, the strength of attachment of the transformer coil to the transformer core is considered sufficient. When the inventors experimentally produced an ultrasound probe of a mechanical sector scanning type using a rotary transformer, however, it became apparent that noise appeared in diagnostic images. In view of the above, the inventors paid attention to the rotary transformer forming part of the signal transfer path in an ultrasound probe, and made intensive studies. As a result, the inventors discovered that noise appeared, because the transformer coil forming a rotary transformer is affected by flux leakage from the magnet of a drive motor and vibrates at a time of transmission/reception of ultrasound waves. An ultrasound probe according to an embodiment of the present invention has been completed on the basis of the above findings.

Hereinafter, an embodiment of the present invention will be described in detail with reference to the drawings. However, the scope of the invention is not limited to the illustrated examples.

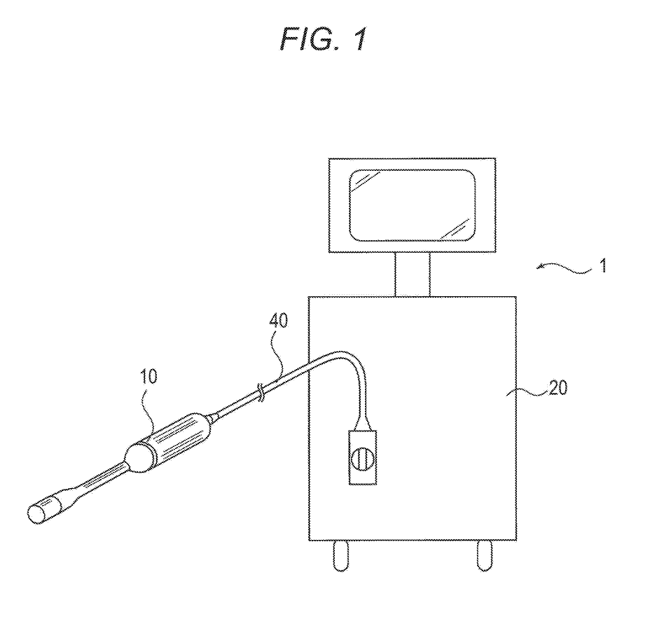

FIG. 1 is an external view of an ultrasound diagnosis apparatus 1 according to an embodiment of the present invention. As shown in FIG. 1, the ultrasound diagnosis apparatus 1 includes an ultrasound probe 10 and a main diagnosis device 20. The ultrasound probe 10 is a transvaginal probe to be inserted into the vagina for observing a fetus, for example. The ultrasound probe 10 transmits ultrasound waves, receives reflected ultrasound waves, converts the reflected ultrasound waves into ultrasound signals, and transmits the ultrasound signals to the main diagnosis device 20. The main diagnosis device 20 supplies a drive signal to the ultrasound probe 10, and generates and displays a diagnostic image in accordance with the received ultrasound signals.

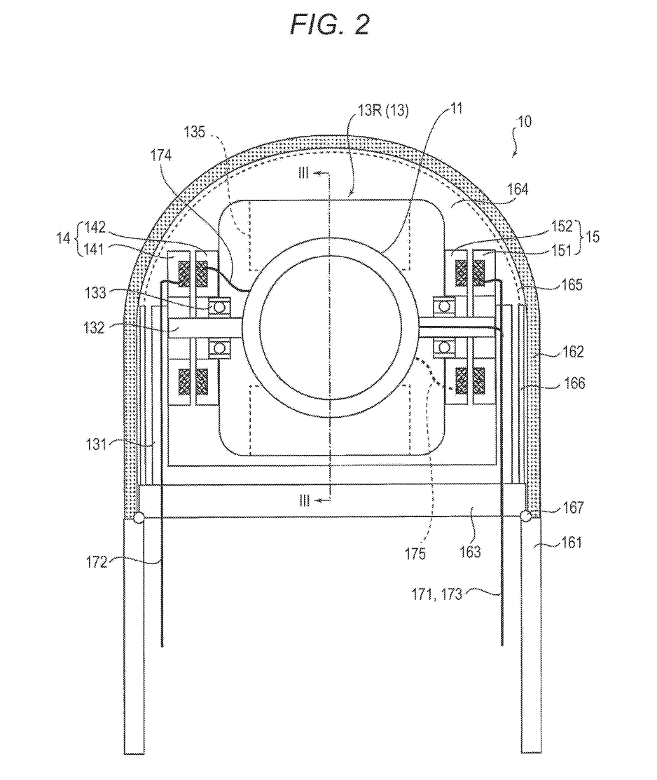

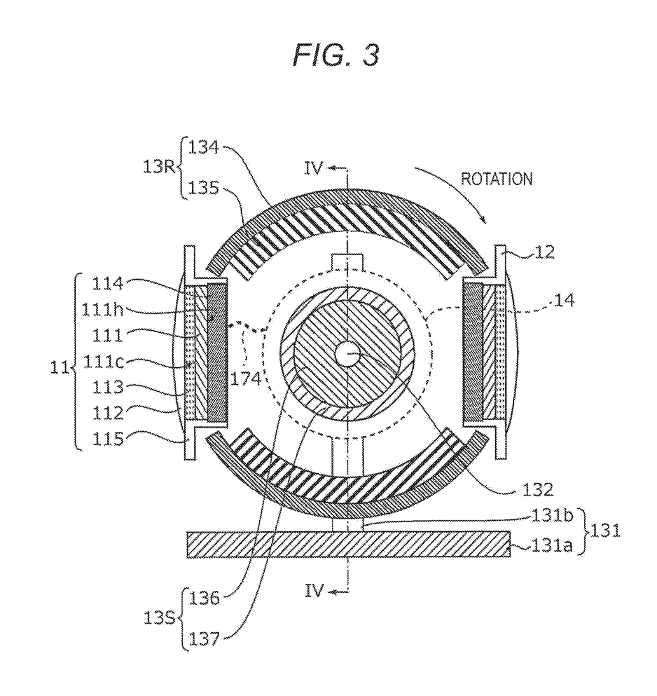

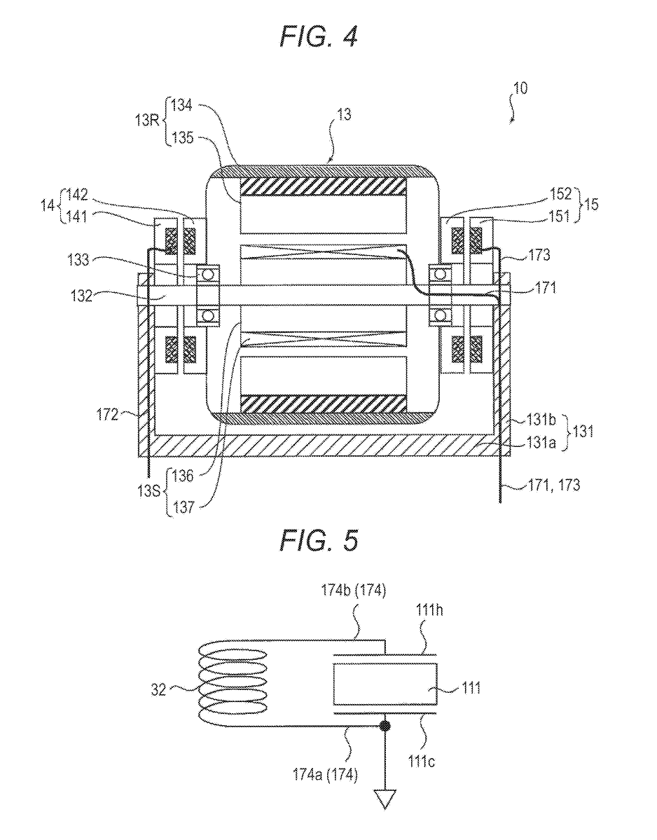

FIG. 2 is a diagram schematically showing the structure of the tip portion of the ultrasound probe 10. FIGS. 3 and 4 are diagrams showing the inner structure of the tip portion of the ultrasound probe 10. FIG. 3 is a cross-sectional view of the tip portion of the ultrasound probe 10, taken along the line defined in FIG. 2. FIG. 4 is a cross-sectional view of the tip portion of the ultrasound probe 10, taken along the line IV-IV defined in FIG. 3.

As shown in FIGS. 2 through 4, the ultrasound probe 10 includes ultrasound elements 11 and 12, a drive motor 13, rotary transformers 14 and 15, and a probe cable 40. The ultrasound probe 10 is a mechanical sector scanning probe. In this embodiment, the ultrasound probe 10 includes the two ultrasound elements 11 and 12. However, the number of ultrasound elements is not limited to any particular number.

In the ultrasound probe 10, a window case 162 is attached one end of a housing 161. The tip portion of the window case 162 has a spherical surface. A frame 163 is disposed between the housing 161 and the window case 162. The frame 163 is connected to the ground wire of the probe cable 40. A sealing member 167, such as an O-ring, is interposed between the housing 161 and the frame 163. With the frame 163, the tip portion of the ultrasound probe 10 is partitioned in a hermetically closed state.

The ultrasound elements 11 and 12, the drive motor 13, the rotary transformers 14 and 15, and the like are disposed in the space 164 formed by the window case 162 and the frame 163. This space 164 is filled with an acoustic coupling liquid.

The ultrasound elements 11 and 12 are attached to the outer peripheral surface of a rotor 13R of the drive motor 13, and rotate with the rotor 13R. The directions of transmission and reception of ultrasound waves at the ultrasound elements 11 and 12 match a radial direction of the rotor 13R. In a case where two or more ultrasound elements are disposed, the ultrasound elements are preferably arranged at regular intervals in a circumferential direction. Since the two ultrasound elements 11 and 12 are attached to the rotor 13R in this embodiment, the ultrasound elements 11 and 12 face each other, with the rotary shaft 132 of the drive motor 13 being interposed in between.

In this example, the ultrasound element 11 is a high-frequency ultrasound element (with a center frequency of 5 to 10 for example), and the other ultrasound element 12 is a low-frequency ultrasound element (with a center frequency of 2.5 to 6 MHz, for example). The ultrasound elements 11 and 12 can be switched in accordance with diagnostic purposes.

In a case where a site with small ultrasound attenuation and at small depth is examined, for example, the high-frequency ultrasound element 11 that can acquire high-resolution images is used. In a case where a site at great depth is examined, the low-frequency ultrasound element 12 that is hardly affected by attenuation. With the single ultrasound probe having such a structure, high-resolution diagnostic images and great-depth diagnostic images can be acquired.

In a case where two or more ultrasound elements are provided, these ultrasound elements may have the same center frequency. As the same diagnostic site is scanned with two or more ultrasound elements, the frame rate or the image update rate can be increased. Thus, an image of an organ that is quick in movement can be displayed in real time.

The ultrasound elements 11 and 12 each include an acoustic lens 112, an acoustic matching layer 113, a piezoelectric plate 111 (a transducer), and a backing material 114, in this order from the side to be brought into contact with a living organism. In FIG. 3, only the components of the ultrasound element 11 are denoted with reference numerals. However, the other ultrasound element 12 has the same structure as the ultrasound element 11. The acoustic matching layer 113, the piezoelectric plate 111, and the backing material 114 are housed in a housing case 115, and the acoustic lens 112 is disposed at the opening end of the housing case 115.

The piezoelectric plate 111 has electrodes 111h and 111c on the transmission/reception surface (the front surface) and the back surface. The piezoelectric plate 111 generates ultrasound waves by vibrating when a drive voltage (the drive signal) is applied thereto from the main diagnosis device 20. The piezoelectric plate 111 also receives ultrasound waves reflected from the inside of a living organism, and converts the reflected ultrasound waves into voltage (ultrasound signals).

The acoustic lens 112 is designed to collect ultrasound waves. The acoustic matching layer 113 is designed to prevent or reduce reflection of ultrasound waves due to a difference in acoustic impedance between the piezoelectric plate 111 and the living organism, and cause ultrasound waves to efficiently propagate in the living organism. The backing material 114 is designed to absorb excess vibration of the piezoelectric plate 111. With these components, resolution performance is improved, and high-definition images can be obtained.

Not only ultrasound waves reflected from the inside of the living organism, but also external electromagnetic waves via the air or the living organism enter the piezoelectric plate 111. These electromagnetic waves appear as noise superimposed on a diagnostic image, and hinder drawing of a high-definition image. Particularly, in a case where signal transfer is performed with the rotary transformers 14 and 15, the ultrasound elements 11 and 12 are put into an electrically floating state, and therefore, are likely to be affected by external electromagnetic waves.

In view of this, the electrode 111c disposed on the front surface of the piezoelectric plate 111 preferably serves as a ground electrode (a cold electrode), and the electrode 111h preferably serves as a signal electrode (a hot electrode) (the electrode 111c and the electrode 111h will be hereinafter referred to as the “ground electrode 111c” and the “signal electrode 111h”, respectively) As shown in FIG. 5, the ground electrode 111c and the signal electrode 111h are electrically connected to lead wires 174a and 174b, respectively. The lead wires 174a and 174b extend from a transformer coil 32 of the rotary transformer 14 (or the rotary transformer 15). The connecting portion between the ground electrode 111c and the lead wire 174a is electrically connected to a conductive rotor core 134 with a screw or a conductive adhesive, and is grounded. With this structure, noise due to external electromagnetic waves can be reduced.

Further, it is beneficial that the ground electrode 111c of the piezoelectric plate 111 is connected to the main diagnosis device 20 with a low impedance. For example, the ground electrode 111c is electrically connected to the rotor core 134 with a conductive adhesive or the like, so that the ground electrode 111c is connected to the ground of the main diagnosis device 20 with a low impedance via the rotor 13R, the rotary shaft 132, a motor base 131, the frame 163, and the probe cable 40.

To reduce influence of external electromagnetic waves, a shield 165 is preferably provided in the window case 162. The shield 165 is a conductive thin film, for example, and is formed by metallic foil application, conductive paint application, plating, vapor deposition, sputtering, or the like. A thin film formed by plating, vapor deposition, or sputtering is particularly preferable, because such a thin film can be very thin, and the influence thence on ultrasound propagation can be reduced. The shield 165 may be formed on the outer surface or the inner surface of the window case 162, or may be buried in the window case 162. To prevent detachment in practical use and take advantage of the simple manufacturing process, the shield 165 is preferably formed on the inner surface of the window case 162.

The shield 165 is electrically connected to a metal plate 166 inserted into and molded in the frame 163, for example. The metal plate 166 is made of aluminum, which is a conductive material, for example. With this arrangement, the shield 165 is connected to the ground of the main diagnosis device 20 with a low impedance via the metal plate 166, the frame 163, and the probe cable 40. Thus, the influence of external electromagnetic waves can be effectively reduced.

The drive motor 13 is a motor of an outer rotor type, including a stator 13S and the rotor 13R. The stator 13S has a structure in which a motor coil 137 is formed on the peripheral surface of a stator core 136. The rotor 13R has a structure in which a motor magnet (a permanent magnet) 135 is attached to the rotor core 134. The stator 13S and the rotor 13R are attached to the motor base 131, and are integrally mounted on the frame 163.

The motor base 131 includes a bottom plate 131a and a supporting plate 131b standing from the bottom plate 131a. The motor base 131 is made of a conductive material, and is grounded via the frame 163. The rotary shaft 132 is secured to upper portions of the supporting plate 131b. The rotary shaft 132 is made of a conductive material, and is grounded via the motor base 131.

The stator 13S is secured to substantially the center of the rotary shaft 132. The rotor 13R is disposed in such a manner as to surround the stator 13S, and is secured to the rotary shaft 132 via bearings 133. The rotor core 134 is grounded via the rotary shaft 132 and the motor base 131.

The rotary transformer 14 is a high-frequency transformer that forms part of the signal transfer path of the ultrasound element 11. The rotary transformer 15 is a low-frequency transformer that forms part of the signal transfer path of the ultrasound element 12. The rotary transformers 14 and 15 have transform characteristics suitable for the center frequencies of the ultrasound elements 11 and 12, respectively. The transform characteristics can be adjusted by changing the number of turns of the transformer coil 32 (see FIG. 5), for example.

The rotary transformers 14 and 15 are preferably disposed on both sides of the drive motor 13 in the axial direction. With this arrangement, the distance between the signal transfer path of the ultrasound element 11 and the signal transfer path of the ultrasound element 12 becomes longer than that in a case where the two rotary transformers 14 and 15 are disposed on one side of the drive motor 13 in the axial direction. Thus, crosstalk can be reduced. In this case, diagnostic images can be acquired wdth the ultrasound element 11 and/or the ultrasound element 12, the center frequencies of the ultrasound element 11 and the ultrasound element 12 being different from each other.

The rotary transformers 14 and 15 include primary transformers 141 and 151, and secondary transformers 142 and 152, respectively. The primary transformers 141 and 151 are secured to the supporting plate 131b of the motor base 131. Signal wires 172 and 173 extended from the probe cable 40 are connected to the primary transformers 141 and 151, respectively. The secondary transformers 142 and 152 are secured to the rotor 13R (a side surface of the rotor core 134) of the drive motor 13. The secondary transformers 142 and 152 are connected to the ultrasound elements 11 and 12 via lead wires 174 and 175, respectively. The secondary transformers 142 and 152 rotate with the rotor 13R.

The rotary transformers 14 and 15 are preferably of a planar opposed type, with the primary transformers 11 and 151 being located on the opposite side from the secondary transformers 142 and 152 in the axial direction of the drive motor 13. With this arrangement, the length in the axial direction can be reduced, and the ultrasound probe 10 can be made smaller in size.

The probe cable 40 is a cable connected to the main diagnosis device 20, and includes a motor wire 171 for the drive motor 13, the signal wires 172 and 173 for the ultrasound elements 11 and 12, and a ground wire (not shown). The motor wire 171 and the signal wires 172 and 173 are introduced into the space 164 at the tip portion through insertion holes in the frame 163 and the motor base 131.

The motor wire 171 is further extended through an insertion hole in the rotary shaft 132, and is connected to the motor coil 137. The signal wires 172 and 173 are connected to the primary transformers 141 and 151, respectively. The portions of the motor wire 171 and the signal wires 172 and 173 introduced into the acoustic coupling liquid are sealed with an adhesive or the like so that the acoustic coupling liquid will not leak out.

When current flows into the motor coil 137 via the motor wire 171, the rotor 13R rotates about the rotary shaft 132. The ultrasound elements 11 and 12 and the primary transformers 141 and 151 secured to the rotor 13R also rotate with the rotor 13R. As ultrasound signals are transmitted with the rotary transformers 14 and 15 in a non-contact manner, rotational scanning of the ultrasound elements 11 and 12 can be performed.

In the ultrasound probe 10, ultrasound waves transmitted from the ultrasound elements 11 and 12 propagate in the acoustic coupling liquid filling the space 164 and in the window case 162. The ultrasound waves then reach the inside of the living organism in contact with the ultrasound probe 10. The ultrasound waves are reflected by boundaries with different acoustic impedances from each other in the living organism, and return to the ultrasound elements 11 and 12 via the window case 162 and the acoustic coupling liquid in the order reversed from the order at the time of transmission. The ultrasound waves received by the ultrasound elements 11 and 12 are then converted into ultrasound signals, and are transmitted to the main diagnosis device 20 via the secondary transformers 142 and 152, the primary transformers 141 and 151, and the signal wires 172 and 173.

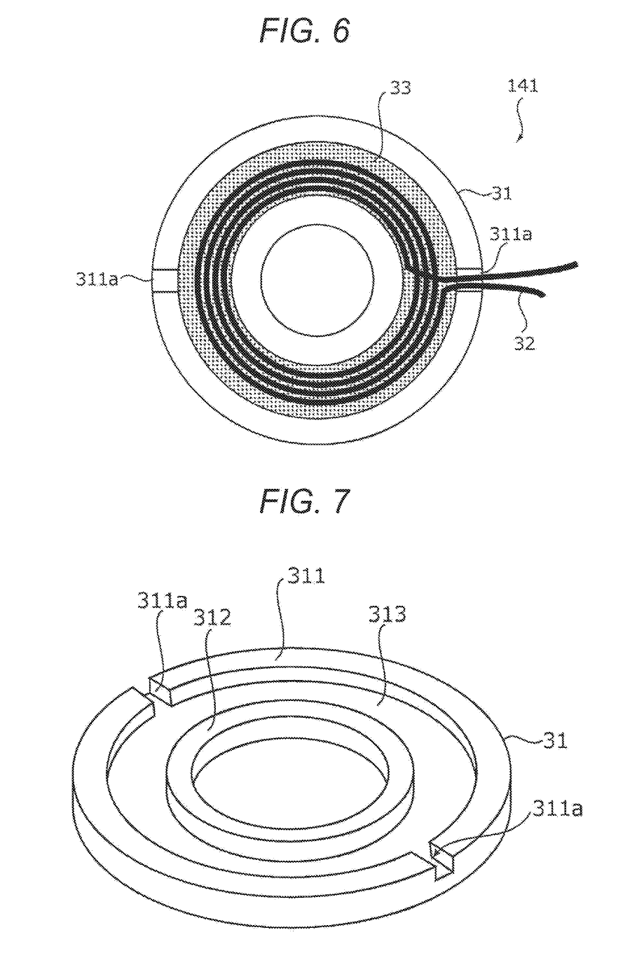

In a case where the rotary transformers 14 and 15 are attached to the rotor 13R, and are located near the motor magnet 135 as in the ultrasound probe 10, the rotary transformers 14 and 15 are affected by flux leakage from the motor magnet 135. Specifically, current flows into the transformer coil 32 (see FIG. 6) of each of the rotary transformers 14 and 15 at the time of ultrasound wave transmission/reception, and at this point of time, electromagnetic force is generated by virtue of the interaction between flux leakage (a magnetic field) and the current in a case where the transformer coil is not firmly secured to the transformer core as in a conventional rotary transformer, the transformer coil vibrates due to the above mentioned electromagnetic force, and current is generated by the interaction between the vibration of the transformer coil and flux leakage. As a result, noise might appear in a diagnostic image.

In view of the above, the winding state of the transformer coil 32 does not change in each of the rotary transformers 14 and 15 in the ultrasound probe 10 of this embodiment. The “winding state of the transformer coil 32” also indicates the positional relationship between the loops of the winding wire forming the transformer coil 32, or the positional relationship between the transformer core 31 and the transformer coil 32.

FIG. 6 is a plan view of the primary transformer 141. FIG. 7 is a perspective view of the transformer core 31. Although an example of the primary transformer 141 is shown in FIG. 6, the primary transformer 151 and the secondary transformers 142 and 152 each have the same fundamental structure as the primary transformer 141.

As shown in FIG. 6, the primary transformer 141 is a transformer in a disk-like shape, and includes the transformer core 31 and the transformer coil 32.

The transformer core 31 has a groove 313 formed by an outer edge portion 311 and an inner edge portion 312 protruding from the toric bottom surface. The groove 313 serves as a coil housing portion that houses the transformer coil 32. A cut-away part 311a for pulling out the transformer coil 32 is also formed in the outer edge portion 311. One cut-away part 311a may suffice, but two cut-away parts 311a may be formed, for example.

The transformer core 31 is made of a Ni—Zn ferritic material, for example. To efficiently transfer electrical signals, a core material with a high initial permeability and a high saturation flux density is required. Candidates for a material having such characteristics include a manganese-ferritic material. However, a Ni—Zn ferritic material is preferable, in terms of frequency characteristics. As the transformer core 31 is made of a Ni—Zn ferritic material, signal transfer can be appropriately performed at 1 to 12 MHz, which is the frequency band of the medical ultrasound probe 10.

The transformer coil 32 is formed by winding a winding wire (a magnet wire) around the inner edge portion 312 of the transformer core 31 a predetermined number of turns. The number of turns of the transformer coil 32 is appropriately adjusted to satisfy the transform, characteristics required in each of the transformers (the primary transformers 141 and 151, and the secondary transformers 142 and 152). For example, when the number of turns of the transformer coil 32 is increased, the inductance becomes greater, and the frequency characteristics of the ultrasound element have a peak on the low-frequency side.

To lower production costs and improve workability in manufacturing (or prevent failed attachment), the primary transformers 141 and 151, and the secondary transformers 142 and 152, may have the same structure, as well as the same number of turns of the transformer coil 32.

In this embodiment, the transformer coil 32 is secured to the transformer core 31 with a coil holding portion 33, so that the winding state will not change. That is, in the rotary transformers 14 and 15, the relative positions of the loops of the winding wire forming the transformer coils 32 do not change. In each of the rotary transformers 14 and 15, the relative positions of the transformer core 31 and the transformer coil 32 do not change, either. With this, the winding states of the transformer coils 32 do not change, and the transformer coils 32 do not vibrate, either, even if electromagnetic force is generated due to the interaction between flux leakage from the motor magnet 135 and the current flowing in the transformer coils 32 at a time of transmission/reception of ultrasound waves. Thus, current other than the current corresponding to ultrasound signals, or current that will cause noise, does not flow in the ultrasound probe 10.

The coil holding portion 33 may be formed with a molding resin, such as epoxy resin. As a molding resin is introduced into the groove 313 and fills the groove 313 while the transformer coil 32 is disposed in the groove 313 of the transformer core 31, the transformer coil 32 can be secured to the groove 313 over the entire perimeter. That is, the transformer core 31 has the groove 313 in which the transformer coil 32 is disposed, arid the coil holding portion 33 buries the transformer coil 32 in the groove 313 with a filler. By this method, the transformer coil 32 can be certainly and firmly secured over the entire perimeter, regardless of the winding state of the transformer coil 32. This method is particularly preferable in a case where the transformer coil 32 has two or more turns.

Alternatively, the coil holding portion 33 may be formed with an adhesive or double-faced adhesive tape, for example. An adhesive is applied to or double-faced adhesive tape is attached to the entire circumference of the groove 313 of the transformer core 31, and the transformer coil 32 is disposed on the adhesive or the double-faced adhesive tape. In this manner, the transformer coil 32 can be secured over the entire perimeter. That is, the coil holding portion 33 attaches the transformer coil 32 to the transformer core 31 over the entire perimeter. If the transformer coil 32 has a single turn, the transformer coil 32 can be easily secured over the entire perimeter by this method. If the transformer coil 32 has two or more turns, the loops of the winding wire are welded to one another or are bonded to one another with an adhesive, to integrate the transformer coil 32.

In a case where an adhesive or double-faced adhesive tape is used as the coil holding portion 33, the adhesive may not be continuously applied, or the double-faced adhesive tape may not be continuously attached to the entire circumference of the groove 313. Instead, the adhesive may be intermittently applied or the double-faced adhesive tape may be intermittently attached to the entire circumference of the groove 313 in such a manner that the winding state of the transformer coil 32 will not change.

As described above, the ultrasound probe 10 includes: the drive motor 13 of an outer rotor type; the ultrasound elements 11 and 12 that transmit ultrasound waves toward an object, and receive ultrasound waves reflected by the object; and the rotary transformers 14 and 15 that are disposed at the ends of the drive motor 13 in the direction of rotational axis, and transfer signals of the ultrasound elements 11 and 12 in a non-contact manner. The drive motor 13 includes: the stator 13S including the stator core 136 and the motor coil 137; and the rotor 13R including the rotor core 134 and the motor magnet 135. The ultrasound elements 11 and 12 are attached to the rotor 13R. The rotary transformers 14 and 15 include the secondary transformers 142 and 152 disposed on the rotor 13R, and the primary transformers 141 and 151 disposed on the opposite side from the secondary transformers 142 and 152. The primary transformers 141 and 151, and the secondary transformers 142 and 152 each include the transformer core 31 and the transformer coil 32 disposed on the transformer core 31. The primary transformers 141 and 151, and the secondary transformers 142 and 152 each include the coil holding portion 33 that maintains the winding state of the transformer coil 32.

The ultrasound diagnosis apparatus 1 includes: the above described ultrasound probe 10; and the main diagnosis device 20 that is connected to the ultrasound probe 10, supplies a drive signal to the ultrasound probe 10, and generates a diagnostic image in accordance with an ultrasound signal from the ultrasound probe 10.

With the ultrasound probe 10, noise due to electromagnetic force generated by the interaction between flux leakage from the motor magnet 135 and the current flowing in the transformer coil 32 can be reduced. Thus, high-definition diagnostic images with less noise can be obtained in the ultrasound diagnosis apparatus 1 including the ultrasound probe 10.

Although the present invention made by the inventor(s) has been described in detail based on an embodiment, the present invention is not limited to the above described embodiment, and changes may be made to the embodiment without departing from the scope of the invention.

For example, the prmary transformers 141 and 151, and the secondary transformers 142 and 152 each preferably include the coil holding portion 33, and the winding states of each of the primary transformers 141 and 151, and the secondary transformers 142 and 152 preferably do not change. However, long as at least one of the transformers includes the coil holding portion 33, noise in a diagnostic image can be made smaller than in the case of a conventional structure.

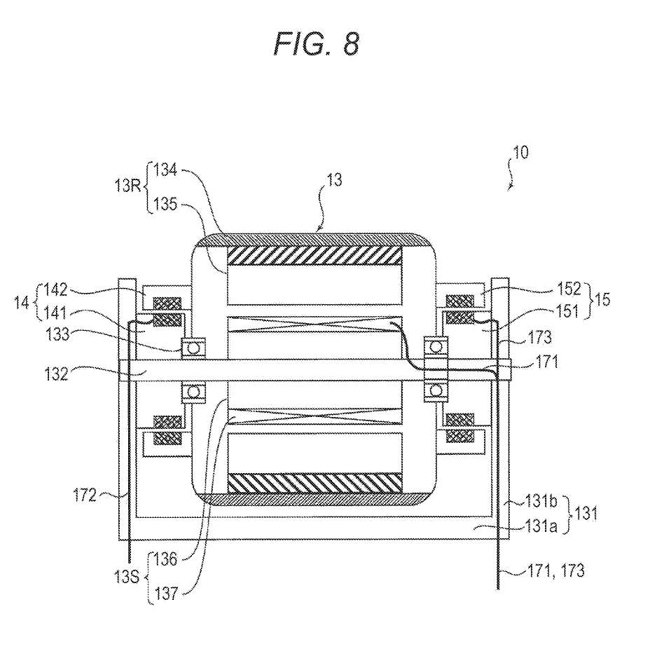

As shown in FIG. 8, coaxial transformers may be used as the rotary transformers 14 and 15. In this case, the primary transformers 141 and 151 are secured to the motor base 131 and are located inside in the radial direction, and the secondary transformers 142 and 152 are secured to the rotor 13R and are located outside the radial direction. The transformer coils 32 of the primary transformers 141 and 151, and the transformer coils 32 of the secondary transformers 142 and 152 are located on the opposite sides in the radial direction.

Although the present invention has been described and illustrated in detail, it is clearly understood that the same is by way of illustrated and example only and is not to be taken by way of limitation, the scope of the present invention being interpreted by terms of the appended claims. It should be understood that equivalents of the claimed inventions and all modifications thereof are incorporated herein.

What is claimed is:

1. An ultrasound probe comprising:
a drive motor of an outer rotor type;
an ultrasound element configured to transmit ultrasound waves toward an object, and receive ultrasound waves reflected by the object; and
a rotary transformer configured to transfer a signal of the ultrasound element in a non-contact manner, the rotary transformer being disposed at an end of the drive motor in a direction of rotational axis,
wherein
the drive motor includes: a stator including a stator core and a motor coil; and a rotor including a rotor core and a motor magnet,
the ultrasound element is attached to the rotor,
the rotary transformer includes a secondary transformer disposed on the rotor, and a primary transformer disposed on the opposite side from the secondary transformer,
the primary transformer and the secondary transformer each include a transformer core, and a transfomer coil disposed on the transformer core, and
at least one of the primary transformer and the secondary transformer includes a coil holding portion configured to maintain a winding state of the transformer coil.

2. The ultrasound probe according to claim 1, wherein relative positions of loops of a winding wire forming the transformer coil do not change.

3. The ultrasound probe according to claim 1, wherein relative positions of the transformer core and the transformer coil do not change.

4. The ultrasound probe according to claim 1, wherein the coil holding portion attaches the transformer coil to the transformer core over an entire perimeter.

5. The ultrasound probe according to claim 1, wherein
the transformer core has a groove in which the transformer coil is disposed, and
the coil holding portion buries the transformer coil in the groove with a filler.

6. The ultrasound probe according to claim 1, wherein both the primary transformer and the secondary transformer include the coil holding portion.

7. The ultrasound probe according to claim 1, wherein the rotary transformer is a transformer of a planar opposed type, with the primary transformer being located on the opposite side from the secondary transformer in the direction of rotational axis of the drive motor.

8. The ultrasound probe according to claim 1, comprising
two of the ultrasound elements,
wherein two of the rotary transformers corresponding to the two ultrasound elements are disposed at both ends of the drive motor in the direction of rotational axis.

9. The ultrasound probe according to claim 8, wherein the number of turns of the two rotary transformers is set in accordance with characteristics of the two ultrasound elements to which the two rotary transformers are connected.

10. The ultrasound probe according to claim 9, wherein
the two ultrasound elements are a high-frequency ultrasound element and a low-frequency ultrasound element, and
the number of turns of the rotary transformer corresponding to the high-frequency ultrasound element is smaller than the number of turns of the rotary transformer corresponding to the low-frequency ultrasound element.

11. The ultrasound probe according to claim 1, wherein the ultrasound element includes: a piezoelectric plate; a ground electrode disposed on a front surface of the piezoelectric plate, the front surface serving as a surface for transmitting and receiving ultrasound waves; and a signal electrode disposed on a back surface of the piezoelectric plate.

12. The ultrasound probe according to claim 11, wherein the ground electrode is electrically connected to the rotor.

13. The ultrasound probe according to claim 1, wherein
the drive motor, the ultrasound element, and the rotary transformer are disposed in a window case filled with an acoustic coupling liquid, and
a shield is provided in the window case.

14. The ultrasound probe according to claim 13, wherein the shield is formed by one of a plating technique, a vapor deposition technique, and a sputtering technique.

15. The ultrasound probe according to claim 13, further comprising
a frame configured to hermetically close the window case, the frame being connected to a ground of a main diagnosis device via a ground wire of a probe cable,
wherein the shield is electrical ly connected to a metal plate inserted into and molded in the frame.

16. An ultrasound diagnosis apparatus comprising:
the ultrasound probe according to claim 1; and
a main diagnosis device configured to supply a drive signal to the ultrasound probe, and generate a diagnostic image in accordance with an ultrasound signal from the ultrasound probe, the main diagnosis device being connected to the ultrasound probe.
